(19) United States
(12) Patent Application Publication
(10) Pub. No.: US 2018/0088049 A1
(43) Pub. Date: Mar. 29, 2018

(54) METHODS AND DEVICES FOR PHOTOMETRIC ANALYSIS

(71) Applicant: AZURE BIOSYSTEMS, INC., Dublin, CA (US)

(72) Inventor: Diping Che, San Ramon, CA (US)

(73) Assignee: AZURE BIOSYSTEMS, INC., Dublin, CA (US)

(21) Appl. No.: 15/715,165

(22) Filed: Sep. 26, 2017

Related U.S. Application Data
(63) Continuation of application No. 62/399,612, filed on Sep. 26, 2016.

(51) Int. Cl. G01N 21/64; G01N 21/76; C12Q 1/68

(52) U.S. Cl. CPC G01N 21/6428; G01N 21/76; C12Q 1/6876; G01N 2201/0636; G01N 2201/06113; G01N 2021/6463; G01N 21/645

(57) ABSTRACT

The present disclosure provides methods and devices for characterizing light-generating analytes.

### PRIORITY CLAIM

This application claims priority to U.S. Provisional Patent Application Ser. No. 62/399,612, filed Sep. 26, 2016, the entirety of which is incorporated herein by reference and relied upon.

### BACKGROUND

Since their introduction, fluorescent and chemiluminescent assays have become fundamental tools in biological research. They quickly replaced much of the hazardous radiolabeling in existing DNA, RNA and protein assays, as well as enabling a level of multiplexing and parallel reactions that radioactivity could never achieve. This provided a critical step needed in order to complete the human genome project, and to support nearly all drug development, gene and protein research, thus paving the way for the modern biologic era.

However, while these methods have made a tremendous increase in the speed and complexity of assays, inefficiencies in imaging resolution and sensitivity limit their utility. Current devices available for these analyses suffer from reduced sensitivity and resolution. For example, most analyzers use sensors that are smaller than the substrate being analyzed; a lens is therefore typically placed a distance from the substrate. However, the greater the distance between the lens and the substrate, the less light can be collected because the light is emitted in all directions, and not just towards the lens. Capturing less light, along with other distance-related issues such as chromatic aberrations, contributes to the low sensitivity of existing analyzers.

Throughout the years, many attempts have been made to improve resolution and sensitivity. Improved lenses add some level of improvement, but cannot make up for the fact that signal will always decrease as the square of the distance the lens or sensor is from the substrate. Increasing the dwell time can offer some additional signal, but fluorescence and chemiluminescent signals quickly become non-linear due to photobleaching of the fluorophore or substrate/peroxide depletion in the case of chemiluminescence. Fluorescent scanners can offer some improvement, but only with added cost, complexity and imaging time.

A need therefore continues to exist for improved biological substrate analyzers and methods of analyzing biological substrates. The present disclosure meets this need.

SUMMARY

The invention provides for a device that enables analysis of biological analytes spatially resolved along a substrate. In some embodiments, these analytes will be spatially resolved in 2 dimensions, for example, in a gel, blot or microtiter plate and the like. In some embodiments analytes may be spatially resolved in 1 dimension, such as in a capillary, or microfluidic channel and the like.

In some embodiments, the sensor will be in intimate contact with the substrate. In some embodiments, a gap of 1 cm or less will be left between the sensor and the substrate. This gap may be left empty or in some embodiments, chemiluminescence, fluorogenic, or other reagents may be delivered to the substrate. In some embodiments fluid contains luminol and peroxide will be flowed over the substrate while it is within a holder. In some embodiments reagents will be placed on substrate before the holder is assembled. In some embodiments, reagents will be delivered to the substrate after the holder is assembled.

In some embodiments, a mirror will be positioned above the substrate to increase the sensitivity of the assay by reflecting light back onto the sensor.

In some embodiments, the present disclosure provides a detection instrument comprising a base including a sensor, and a lid including a light source and defining a cavity, wherein the cavity is sized to accommodate a biological substrate.

In other embodiments, the present disclosure provides a method of analyzing a biological substrate, the method comprising: inserting a biological substrate in a cavity of a detection instrument, the detection instrument further comprising a sensor in optical communication with the cavity, and a light source in optical communication with the sensor and positioned opposite the cavity from the sensor; illuminating the biological substrate at a first wavelength with the light source; collecting emitted light from the biological substrate at a second wavelength with the sensor; optionally collecting emitted light from the biological substrate at a third wavelength with the sensor; and quantifying the collected emitted light from the second wavelength and optional third wavelength.

### BRIEF DESCRIPTION OF THE DRAWINGS

FIG. 1 shows a detection instrument consistent with one embodiment of the present disclosure.

FIG. 2A shows a side view or cross-sectional view of a sensor-substrate-sensor assembly consistent with one embodiment of the present disclosure.

FIG. 2B shows a comparison of edge effects when pixels of one sensor are aligned with pixels of a second sensor in a sensor-substrate-sensor assembly consistent with FIG. 2A.

FIG. 3A shows a curved sensor suitable for use with a detection instrument as disclosed herein.

FIG. 3B shows a perspective view of a portion of a detection instrument including a curved sensor, such as the curved sensor of FIG. 3A, consistent with one embodiment of the present disclosure.

The figures depict various embodiments of this disclosure for purposes of illustration only. One skilled in the art will readily recognize from the following discussion that alternative embodiments of the structures and methods illustrated herein may be employed without departing from the principles of embodiments described herein.

### DETAILED DESCRIPTION

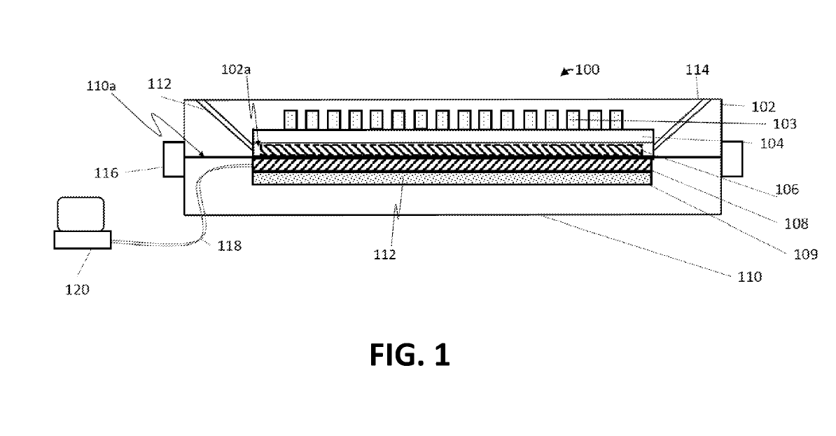

Referring generally to FIGS. 1-3B, the present disclosure provides detection instruments and methods of using same to analyze biological substrates, such as separation gels, nitrocellulose blots, or liquid samples such as those housed in a tube or microtiter well. The detection instruments generally comprise a sensor, a housing, and a light source, but do not include a lens between the biological substrate and the sensor.

Referring now to FIG. 1, a detection instrument 100 according to one embodiment of the present disclosure comprises a base 110, a lid 102, a sensor 108, a light source 103, optionally a mirror 104, optionally a filter 109, and optionally a temperature controller 112.

The base 110 includes the sensor 108 in some embodiments. The sensor 108 may include a single sensor (e.g., a multipixel sensor such as FTF9168M, (Teledyne DALSA, Waterloo, ON, Canada) or OTFT+OPD array on plastic (Isorg, Grenoble, France), or may include multiple individual sensors arranged in an array. In some embodiments, the base 110 includes a filter 109 over the top surface of the sensor 108 to reduce an amount of one or more wavelengths of light from reaching the sensor 108. In some embodiments, the base 110 does not include a lens between the substrate 106 and the sensor 108.

The lid 102 is sized to fit over the base 110 to minimize or even eliminate ambient light from reaching the sensor 108. In some embodiments, such as those in which the top surface of the sensor 108 or the filter 109 is at an elevation at, near or beyond an elevation of the top surface of the base 110a, the lid 102 may include a cavity 102a to accommodate the substrate 106 when the lid 102 is closed onto the base 110.

In some embodiments, the lid 102 includes a light source 103 for illuminating the substrate 106 with one or more wavelengths of light. In some embodiments, the light source 103 includes any one or more of an LED, a laser, an ultraviolet lamp, an infrared lamp, a near-infrared lamp, a UV-Vis lamp, a halogen lamp, an incandescent lamp, a fluorescent lamp, or any other source of light which can irradiate the substrate 106. In some embodiments, the light source 103 comprises at least one laser diode, at least one RGB (red/green/blue) diode, at least one white light lamp (e.g., an EPI white light lamp), and at least one blue LED lamp. In some embodiments, the light source 103 comprises a 658 nm laser diode, a 785 nm laser diode, an RGB LED that emits light at 460 nm, 536 nm, and/or 628 nm, an EPI white light lamp, at least one blue LED that emits light at 470 nm, and at least one ultraviolet lamp that emits light at 302 nm and/or 365 nm. In some embodiments, nm.

In some embodiments, the light source 103 comprises one or more laser lamps (e.g., solid state laser lamps). In some embodiments, the light source 103 includes one laser lamp that emits light at 488 nm, 520 nm, 658 nm, or 785 nm. In some embodiments, the light source 103 includes two laser lamps, wherein a first laser lamp emits light at 488 nm, 520 nm, 658 nm, or 785 nm, and wherein a second laser lamp emits light at a different wavelength than the first laser lamp that is selected from the group consisting of 488 nm, 520 nm, 658 nm, and 785 nm. In some embodiments, the light source 103 includes a first laser lamp that emits light at 658 nm, and a second laser light that emits light at 785 nm. In some embodiments, the light source 103 includes three laser lamps, wherein a first laser lamp emits light at 488 nm, 520 nm, 658 nm, or 785 nm, wherein a second laser lamp emits light at a different wavelength than the first laser lamp that is selected from the group consisting of 488 nm, 520 nm, 658 nm, and 785 nm, and wherein the third laser lamp emits light at a wavelength different from the first laser lamp and the second laser lamp that is selected from the group consisting of 488 nm, 520 nm, 658 nm, and 785 nm. In some embodiments, the light source 103 includes a first laser lamp that emits light at 488 nm, a second laser lamp that emits light at 520 nm, and a third laser lamp that emits light at 658 nm. In some embodiments, the light source 103 includes four laser lamps, wherein one laser lamp emits light at 488 nm, a second laser lamp emits light at 520 nm, a third laser lamp emits light at 658 nm, and a fourth laser lamp emits light at 785 nm.

The light source 103 is in operative communication with a power source (not shown) and optionally with a computer 120 which in some embodiments may be configured to control the light source 103. For example, in embodiments in which the light source 103 includes a laser lamp and an ultraviolet lamp, the computer 120 may be configured to select (a) which of the laser lamp or ultraviolet lamp (or both) are energized, (b) a length of time for which the light source 103 is energized, (c) a length of time for which the light source is 103 is not energized, (d) an intensity (e.g., wattage) at which the light source 103 is energized, (e) a wavelength(s) at which the light source 103 emits light, or (f) any combination of (a)-(e). In some embodiments, the light source 103 is in operative communication with a switch (not shown), for example a mechanical or digital switch, by which a user may manually control (a) which of the laser lamp or ultraviolet lamp (or both) are energized, (b) a length of time for which the light source 103 is energized, (c) a length of time for which the light source is 103 is not energized, (d) an intensity (e.g., wattage) at which the light source 103 is energized, (e) a wavelength(s) at which the light source 103 emits light, or (f) any combination of (a)-(e).

In some embodiments, the lid 102 comprises an inlet 112 and an outlet 114. The inlet 112 and outlet 114 enable a fluid to be added to the substrate 106 without opening the lid 102. In some embodiments, the fluid comprises one or more of: a wash buffer, an antibody, a chemiluminescent reagent, and a fluorogenic reagent.

The mirror 104, when present, may increase the sensitivity of the detection instrument 100 by reflecting emitted light from the substrate 106 back onto the sensor 108. In embodiments wherein the mirror 104 is located between the light source 103 and the substrate 106, such as the embodiment shown in FIG. 1, the mirror 104 may be a dichroic mirror. For example, if the light source 103 comprises a laser emitting light at 658 nm, the mirror 104 may be a dichroic mirror that allows light at 658 nm to pass through, but reflects light emitted by an excited fluorescent label from the substrate 106 towards the sensor 108.

The temperature control 112, when present, may include any suitable active or passive temperature control element known in the art, such as a heat sink, a Peltier device, an electrical heater, or any other controller which can be used to control the temperature of a substrate 106. For example, in some embodiments, the temperature control 112 comprises a thermoelectric block (e.g., a Peltier block) controlled by a controller, such as a thermoelectric controller. The temperature control element in some embodiments is in thermal communication with the substrate 106. In some embodiments, for example, the temperature control element is in conductive thermal communication with the substrate 106 via the sensor 108 and the filter 109, when present.

In some embodiments, the lid 102 is attached to the base 100 by a hinge (not shown).

In some embodiments, a clamp 116 secures the lid 102 to the base 110. In some embodiments, the clamp 116 is placed around at least a portion of the junction between the lid 102 and the base 110, for example to reduce (e.g., minimize) ambient light from entering the cavity 102a.

The filter 109 may be any suitable band-pass filter that enhances detection (e.g., increases a signal-to-noise ratio) of a light-emitting region of a substrate 106, and/or that enables selective detection of different colors emitted by more than one type of fluorescent label. In some embodiments, the filter 109 is selected from the group consisting of: an FRLP filter, an EtBr filter, an IR 780 filter, and a short wave short pass (SWSP) filter.

The sensor 108 may be any suitable emitted light detector. In some embodiments, the sensor 108 is selected from the group consisting of: a charge coupled device (CCD), a cMOs image sensor or area imaging sensors.

While the assembly is shown with lid on top and base on bottom in FIG. 1, this could easily be reversed. For example, in an alternative embodiment, a detection instrument may include a lid comprising a sensor 108 and optionally a filter 109; and a base comprising a light source 103, an optional mirror 104, and an optional inlet 112 and outlet 114.

In some embodiments, computer 120 can receive data from the assembly through wires 118. In some embodiments, computer 120 and assembly 100 will be wirelessly connected over Bluetooth, wifi, or other wireless data transfer options known in the art.

In a representative method of analyzing a substrate 106 using detection instrument 100 consistent with the present disclosure, the substrate 106 is placed on sensor 108 before lid 102 is fastened to base 110. Base 110 and lid 102 can be manufactured out of any material compatible with intended use, and in some embodiments for a light-tight seal protecting the sensor from background room light. In some embodiments, the lid 102 includes a mirror 104 to increase detection sensitivity. In some embodiments, the detection instrument 100 comprises a light source 103 will be contained within the lid, allowing for fluorescent excitation of various wavelength. In some embodiments, mirror 104 is a dichroic mirror that transmits light of a specific wavelength range and reflects light of a specific wavelength range.

In some embodiments, substrate 106 is placed on sensor 108 before lid 102 is fastened to base 110. Base 110 and lid 102 can be manufactured out of any material compatible with intended use, and in some embodiments for a light-tight seal protecting the sensor form background room light. In some embodiments, the lid will contain mirror 104 to increase detection sensitivity. In some embodiments, light source 103 will be contained within the lid, allowing for fluorescent excitation of various wavelength. In some embodiments, mirror 104 will be a dichroic mirror that transmits light of a specific wavelength range and reflects light of a specific wavelength range.

Substrate 106 may be a polyacrylamide gel, agarose gel, nitrocellulose blot, polyvinylidine difluoride membrane (PVDF), microtiter plate, peptide array, protein array, DNA array, microfluidic chip, capillary, ddPCR plate or any other 1 or 2-dimensional substrate used to spatially resolve different biomolecules or reaction conditions. Substrate 106 can be emitting light as a result of fluorescence, chemiluminescence, phosphorescence, bioluminescence, or any other illumination that can be indicative of results for the assay being performed. Sensor 108 can be a digital light measuring device such as charge coupled device (CCD), photomultiplier tube (PMT), photodiode, printed organic photodiode, and the like.

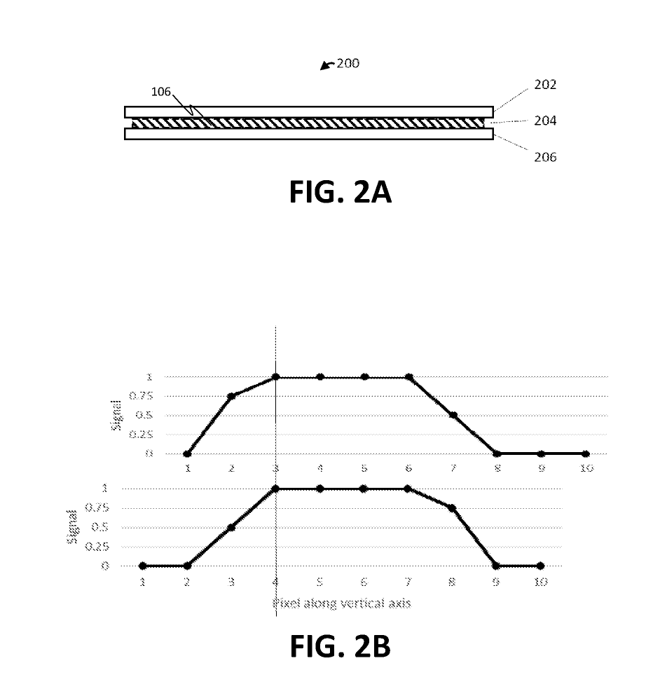

Referring now to FIG. 2A, the present disclosure provides a sensor assembly 200 comprising a substrate gap 204 defined by a first sensor 202 and a second sensor 206. In operation, a substrate 106 may be placed in the substrate gap 204 to form a sensor-substrate-sensor sandwich. In such arrangements, sensor assemblies 200 enable capture of all or substantially all light emitted from the substrate 106. In some embodiments, a computer (not shown) in operative communication with the first sensor 202 and the second sensor 206 is configured to determine an alignment of pixels of the first sensor 202 and pixels of the second sensor, and thereafter to combine emitted light measurements from each pair of aligned pixels from the first sensor 202 and the second sensor 206 to more accurately determine an intensity of light emittance from distinct areas of a substrate 106. FIG. 2B illustrates additive intensities from aligned pixels from sensor 202 (top panel) and sensor 206 (bottom panel).

FIG. 2A discloses a representative diagram according to another embodiment of the invention. Assembly 200 shows a substrate 106 sandwiched between 2 sensors (202 and 206). This configuration not only is able to capture nearly 100% of the emitted light from the substrate 106, but through comparison of edge effects, shown in FIG. 2B, resolution can be measured to greater than the sensor pixel size. By sandwiching the substrate between the two sensors, the spatial relationship of the two sensors is correlated. The pixel intensities can be used to extrapolate the improved resolution. Pixels that are offset in one direction will result in different signal measured in pixels on the two sensors (FIG. 2B). By having the pixel 3, or (m,n), on one sensor 202 (top panel) vertically aligned with pixel 4, or (m,n)′, on the second sensor 206 (bottom panel), the signal product I(m,n)*I′(m,n)′ will construct a modular transfer function, which can effectively double the resolution in the vertical dimension.

Sensors 202 and 206 may each be any suitable emitted light detector. In some embodiments, sensor 202 is the same type as sensor 206. In other embodiments, sensor 202 is a different type than sensor 206. Each of sensor 202 and sensor 206 may be selected from the group consisting of: a charge coupled device (CCD), a photomultiplier tube (PMT), a photodiode, and a printed organic photodiode.

Sensor assemblies 200 consistent with FIG. 2A may be incorporated into a detection instrument 100 consistent with the present disclosure, for example in place of sensor 108 and optional mirror 104 as described herein and/or shown in FIG. 1. Each of sensor 202 and sensor 206 may be in operative communication with a computer (e.g., computer 120 as shown in FIG. 1) via wires (e.g., wires 118) or wirelessly via Bluetooth, WiFi, or other wireless data transfer protocol.

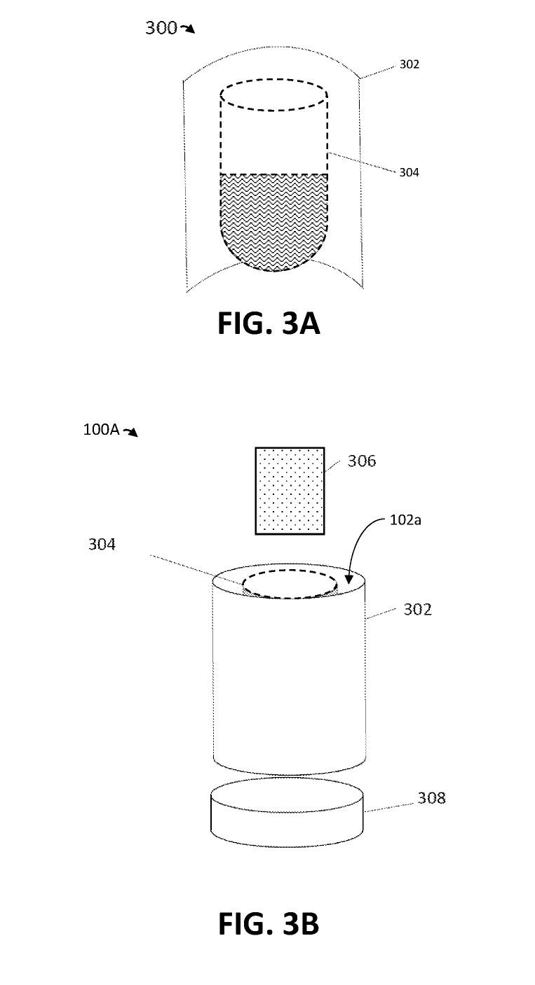

FIG. 3 discloses a representative diagram according to an embodiment of the present disclosure. As shown in FIG. 3A, sensor assembly 300 comprises a curved sensor 302 into which a tube 304 can be placed. Tube 304 contains a fluorophore, a source of chemiluminescence, phosphorescence, bioluminescence, or another illuminating species that can be indicative of results for an assay being performed. FIG. 3B shows the curved sensor 302 wrapped tightly around a tube 304 in a representative detection instrument 100A, thereby being able to collect all or substantially all of the light emitted from the tube 304 for improved quantitation. Light source 306 can be an LED, laser, lamp, or any other source of light which can irradiate tube 304. In some embodiments, the light source 306 is positioned orthogonal to the curvature of the curved sensor 302. For example, as shown in FIG. 3B, the light source 306 illuminates the tube 304 from a position orthogonal to the circular cross section of the curved sensor 302. In such embodiments, light emitting from the light source 306 does not need to pass through the curved sensor 302 to contact the contents of the tube 304.

In some embodiments, the curved sensor 302 comprises an organic photodiode array.

In some embodiments, tube 304 is part of (e.g., one or more wells of) a microtiter plate, or is an array consisting of multiple reactions and detectors.

In some embodiments, the detection instrument 100A further comprises a temperature controller 308 for reducing (e.g., minimizing or eliminating) inaccuracies associated with unsteady sample temperature. Temperature controller 308 can be any suitable active or passive temperature control device, such as a heat sink, a Peltier device, an electrical heater, or any other controller which can be used to control the temperature of tube 304.

In some embodiments, the present disclosure provides a detection instrument 100/100A comprising a base 110 including a sensor 108/200/302, and a lid 102 including a light source 103/306 and defining a cavity 102a or substrate gap 204, wherein the cavity 102a or substrate gap 204 is sized to accommodate a biological substrate 106/304. In some embodiments, the lid 102 further includes a mirror 104. In some embodiments, the mirror 104 is a dichroic mirror. In some embodiments, the detection instrument 100/100A further comprises a filter 109 positioned between the light source 103/306 and the sensor 108/200/302. In some embodiments, the lid 102 further comprises an inlet 112 in fluid communication with the cavity 102a, and an outlet 114 in fluid connection with the cavity 102a. In some embodiments, the inlet 112 is positioned substantially opposite the outlet 104. In some embodiments, the detection instrument 100/100A further comprises a computer 120 in operative communication with the sensor 108/200/302. In some embodiments, the sensor 108/200/302 is selected from the group consisting of: a charge coupled device (CCD), a photomultiplier tube (PMT), a photodiode, and a printed organic photodiode. In some embodiments, the light source 103/306 is one or more of: an LED, a laser, an ultraviolet lamp, an infrared lamp, a near-infrared lamp, a UV-Vis lamp, a halogen lamp, an incandescent lamp, and a fluorescent lamp. In some embodiments, the detection instrument 100/100A further comprises a clamp 116 for securing the lid 102 to the base 110. In some embodiments, the sensor 108/200/302 comprises a sensor assembly 200 comprising a first sensor 202 and a second sensor 206 positioned opposite a substrate gap 204. In some embodiments, the sensor 108/200/302 comprises a curved sensor 302. In some embodiments, the detection instrument 100/100A further comprises a temperature controller 108 in thermal communication with the cavity 102a. In some embodiments, the detection instrument 100/100A does not include a lens between the sensor 108/200/302 and the substrate 106/304.

In some embodiments, the present disclosure provides a method of analyzing a biological substrate 106/304, the method comprising inserting a biological substrate 106/304 in a cavity 102a of a detection instrument 100/100A, the detection instrument 100/100A further comprising a sensor 108/200/304 in optical communication with the cavity 102a, and a light source 103/306 in optical communication with the sensor 108/200/302 and positioned opposite the cavity 102a from the sensor 108/200/302; illuminating the biological substrate 106/304 at a first wavelength with the light source 103/306; collecting emitted light from the biological substrate 106/304 at a second wavelength with the sensor 108/200/302; optionally collecting emitted light from the biological substrate 106/304 at a third wavelength with the sensor 108/200/302; and quantifying the collected emitted light from the second wavelength and optional third wavelength. In some embodiments, the detection instrument 100/100A further comprises a filter 109 positioned between the substrate 106/304 and the sensor 108/200/302. In some embodiments, the detection instrument 100/100A further comprises a mirror 104 positioned between the substrate 106/304 and the light source 103/306. In some embodiments, the biological substrate 106/304 is a PVDF membrane. In some embodiments, biological substrate 106/304 is a chemiluminescent substrate. In some embodiments, the biological substrate 106/304 is a tube. In some embodiments, the sensor 108/200/302 is a curved sensor. In some embodiments, the detection instrument 100/100A does not include a lens between the substrate 106/304 and the sensor 108/200/302.

### EXAMPLES

Aspects of embodiments may be further understood in light of the following examples, which should not be construed as limiting in any way.

### Example 1

### Analysis of Chemiluminescent Western Blot

Cell lysate prepared from HT-29 cell culture grown under standard conditions and treated with 500 ng/mL insulin are lysed in sodium dodecyl sulfate (SDS) containing Laemmle buffer and polyacrylamide gel electrophoresis (SDS-PAGE) is run on a 10% polyacrylamide gel for 1 hour at 1000V. Afterwards, separated proteins are transferred from gel onto PVDF membrane by electroblotting. The membrane is washed and blocked using standard procedures, and incubated with ERK primary antibody (Millipore cat. no. 06-182) at a 1:100 dilution for 2 hours at room temperature. The membrane is washed and then incubated with Horseradish peroxidase (HRP) labeled anti-rabbit secondary antibody (ThermoFisher cat. no. 81-6120) at 1:10,000 dilution for 30 minutes at room temperature. The membrane is then rinsed with buffer and ready to load into detection instrument 100 as shown in FIG. 1.

The membrane is laid down and makes contact with sensor 108, a printed organic photodiode. Filter 109 is not used in chemiluminescent assay and may be removed. Lid 102 is then attached to base 110 using clamp 116. Luminol and peroxide mixture (ThermoFisher cat. no. 34075) is loaded onto the membrane through inlet 112.

HRP degrades luminol at the sites the antibody pair is bound to ERK, producing light. This light emitted downward is detected by sensor 108 directly and light emitted upward is reflected off mirror 104 back onto sensor 108. After 10 seconds, sensor 108 is queried by computer 120 via wire 218, and data from pixel array is recorded and presented to user through a graphical user interface (Azure cSeries software).

### Example 2

### Analysis of Fluorescent Western Blot

Cell lysate prepared from HT-29 cell culture grown under standard conditions and treated with 500 ng/mL insulin are lysed in sodium dodecyl sulfate (SDS) containing Laemmle buffer and polyacrylamide gel electrophoresis (SDSPAGE) is run on a 10% polyacrylamide gel for 1 hour at 1000V. Afterwards, separated proteins are transferred from gel onto PVDF membrane by electroblotting. The membrane is washed and blocked using standard procedures, and incubated with ERK primary antibody (Millipore cat. no. 06-182) at a 1:100 dilution for 2 hours at room temperature. The membrane is washed and then incubated with Cy5 labeled anti-rabbit secondary antibody (ThermoFisher cat. no. 81-1616) at 1:10,000 dilution for 30 minutes at room temperature. The membrane is then rinsed with buffer and ready to load into detection instrument 100 as shown in FIG. 2.

The membrane is laid down and makes contact with filter 109, which is placed on top of sensor 108, a printed organic photodiode. Lid 102 is then attached to base 110 using clamp 116. LEDs 103 are illuminated, and light from LEDs passes through bandpass filter 104 so that only light of wavelength 590-610 nm illuminates the gel. The Cy5 fluorescent stain absorbs the light, and re-emits at 670 nm. Bandpass filter 109, transmits light from 660-680 nm, which is detected by sensor 108. After 1 second, sensor 108 is queried by computer 120 via wire 118, and data from pixel array is recorded and presented to user through a graphical user interface (Azure cSeries software, Azure Biosystems, Inc.; Dublin, Calif.).

### Example 3

### Analysis of Chemiluminescent ELISA

ELISA is prepared using standard protocols known in the art in a clear-bottom 96-well plate. HRP-labeled secondary antibody is added as final antibody step and incubated for 30 minutes at room temperature. The wells are rinsed and loaded with chemiluminescent substrate, and ready to load into detection instrument 100.

The 96-well plate is laid down and makes contact with sensor 108, a printed organic photodiode. Filter 109 is not used in chemiluminescent assay and may be removed. Lid 102 is then attached to base 110 using clamp 116.

HRP degrades luminol at a rate related to amount of target protein in the well. This light emitted downward is detected by sensor 108 directly and light emitted upward is reflected off mirror 104 back onto sensor 108. After 10 seconds, sensor 108 is queried by computer 120 via wire 118, and data from pixel array is recorded and presented to user through a graphical user interface (Azure cSeries software, Azure Biosystems, Inc.; Dublin, Calif.).

### Example 4

### Analysis of Fluorescent Taqman Assay in Microtube

Cell lysate is prepared and mixed with Taqman reagents according to standard protocol, and loaded into a 100 microliter microtube 304. A printed organic photodiode 302 is formed into a cylinder and the microtube 304 is placed inside. Quantitative PCR is then performed by altering tube 304 temperature by cycling the temperature of Peltier device 308 through annealing, extension, and denature temperature points. As the amount of amplified target DNA sequence is increased, fluorescence of taqman probe is detected by sensor 302.

### Further Examples

Further Example 1. A detection instrument comprising:
a base including a sensor; and
a lid including a light source and defining a cavity,

wherein the cavity is sized to accommodate a biological substrate.

Further Example 2. The detection instrument of Further Example 1, wherein the lid further includes a mirror.

Further Example 3. The detection instrument of Further Example 2, wherein the mirror is a dichroic mirror.

Further Example 4. The detection instrument of any preceding Further Example further comprising a filter positioned between the light source and the sensor.

Further Example 5. The detection instrument of any preceding Further Example, wherein the lid further comprises an inlet in fluid communication with the cavity, and an outlet in fluid connection with the cavity.

Further Example 6. The detection instrument of Further Example 5, wherein the inlet is positioned substantially opposite the outlet.

Further Example 7. The detection instrument of any preceding Further Example further comprising a computer in operative communication with the sensor.

Further Example 8. The detection instrument of any preceding Further Example, wherein the sensor is selected from the group consisting of: a charge coupled device (CCD), a photomultiplier tube (PMT), a photodiode, and a printed organic photodiode.

Further Example 9. The detection instrument of any preceding Further Example, wherein the light source is one or more of: an LED, a laser, an ultraviolet lamp, an infrared lamp, a near-infrared lamp, a UV-Vis lamp, a halogen lamp, an incandescent lamp, and a fluorescent lamp.

Further Example 10. The detection instrument of any preceding Further Example further comprising a clamp for securing the lid to the base.

Further Example 11. The detection instrument of any preceding Further Example, wherein the sensor comprises a sensor assembly comprising a first sensor and a second sensor positioned opposite a substrate gap.

Further Example 12. The detection instrument of any preceding Further Example, wherein the sensor comprises a curved sensor.

Further Example 13. The detection instrument of any preceding Further Example further comprising a temperature controller in thermal communication with the cavity.

Further Example 14. The detection instrument of any preceding Further Example, wherein the detection instrument does not include a lens.

Further Example 15. A method of analyzing a biological substrate, the method comprising:
inserting a biological substrate in a cavity of a detection instrument, the detection instrument further comprising:
   a sensor in optical communication with the cavity, and
   a light source in optical communication with the sensor and positioned opposite the cavity from the sensor;
illuminating the biological substrate at a first wavelength with the light source;
collecting emitted light from the biological substrate at a second wavelength with the sensor;
optionally collecting emitted light from the biological substrate at a third wavelength with the sensor; and
quantifying the collected emitted light from the second wavelength and optional third wavelength.

Further Example 16. The method of Further Example 15, wherein the detection instrument further comprises a filter positioned between the substrate and the sensor.

Further Example 17. The method of Further Example 15 or Further Example 16, wherein the detection instrument further comprises a mirror positioned between the substrate and the light source.

Further Example 18. The method of any one of Further Examples 15-17, wherein the biological substrate is a PVDF membrane.

Further Example 19. The method of any one of Further Examples 15-17, wherein the biological substrate is a chemiluminescent substrate.

Further Example 20. The method of any one of Further Examples 15-17, wherein the biological substrate is a tube.

Further Example 21. The method of Further Example 20, wherein the sensor is a curved sensor.

Further Example 22. The method of any one of Further Examples 15-20, wherein the detection instrument does not include a lens.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents.

It is to be understood that both the foregoing descriptions are exemplary and explanatory only, and are not restrictive of the methods and devices described herein. In this application, the use of the singular includes the plural unless specifically stated otherwise. Also, the use of “or” means “and/or” unless stated otherwise. Similarly, “comprise,” “comprises,” “comprising,” “include,” “includes” and “including” are not intended to be limiting.

All patents, patent applications, publications, and references cited herein are expressly incorporated by reference to the same extent as if each individual publication or patent application was specifically and individually indicated to be incorporated by reference.

What is claimed is:

1. A detection instrument comprising:
a base including a sensor; and
a lid including a light source and defining a cavity,
wherein the cavity is sized to accommodate a biological substrate.

2. The detection instrument of claim 1, wherein the lid further includes a mirror.

3. The detection instrument of claim 2, wherein the mirror is a dichroic mirror.

4. The detection instrument of claim 1 further comprising a filter positioned between the light source and the sensor.

5. The detection instrument of claim 1, wherein the lid further comprises an inlet in fluid communication with the cavity, and an outlet in fluid connection with the cavity.

6. The detection instrument of claim 5, wherein the inlet is positioned substantially opposite the outlet.

7. The detection instrument of claim 1 further comprising a computer in operative communication with the sensor.

8. The detection instrument of claim 1, wherein the sensor is selected from the group consisting of: a charge coupled device (CCD), a photomultiplier tube (PMT), a photodiode, and a printed organic photodiode.

9. The detection instrument of claim 1, wherein the light source is one or more of: an LED, a laser, an ultraviolet lamp, an infrared lamp, a near-infrared lamp, a UV-Vis lamp, a halogen lamp, an incandescent lamp, and a fluorescent lamp.

10. The detection instrument of claim 1 further comprising a clamp for securing the lid to the base.

11. The detection instrument of claim 1, wherein the sensor comprises a sensor assembly comprising a first sensor and a second sensor positioned opposite a substrate gap.

12. The detection instrument of claim 1, wherein the sensor comprises a curved sensor.

13. The detection instrument of claim 1 further comprising a temperature controller in thermal communication with the cavity.

14. The detection instrument of claim 1, wherein the detection instrument does not include a lens.

15. A method of analyzing a biological substrate, the method comprising:
inserting a biological substrate in a cavity of a detection instrument, the detection instrument further comprising:
a sensor in optical communication with the cavity, and
a light source in optical communication with the sensor and positioned opposite the cavity from the sensor;
illuminating the biological substrate at a first wavelength with the light source;
collecting emitted light from the biological substrate at a second wavelength with the sensor;
optionally collecting emitted light from the biological substrate at a third wavelength with the sensor; and
quantifying the collected emitted light from the second wavelength and optional third wavelength.

16. The method of claim 15, wherein the detection instrument further comprises a filter positioned between the substrate and the sensor.

17. The method of claim 15, wherein the detection instrument further comprises a mirror positioned between the substrate and the light source.

18. The method of claim 15, wherein the biological substrate is a PVDF membrane, a chemiluminescent substrate, or a tube.

19. The method of claim 18, wherein the biological substrate is a tube and the sensor is a curved sensor.

20. The method of claim 15, wherein the detection instrument does not include a lens.
